(19) United States
(12) Patent Application Publication
(10) Pub. No.: US 2005/0245822 A1
(43) Pub. Date: Nov. 3, 2005

(54) Method and apparatus for imaging distant anatomical structures in intra-cardiac ultrasound imaging

(75) Inventors: Praveen Dala-Krishna, Bensalem, PA (US); Charles Bryan Byrd, Medford, NJ (US); David A. Jenkins, Flanders, NJ (US)

Correspondence Address: HELLER EHRMAN WHITE & MCAULIFFE LLP, 1717 RHODE ISLAND AVE, NW, WASHINGTON, DC, 20036-3001, US

(73) Assignee: EP MedSystems, Inc., West Berlin, NJ (US)

(21) Appl. No.: 11/081,498

(22) Filed: Mar. 17, 2005

Related U.S. Application Data
(63) Continuation-in-part of application No. 10/620,517, filed on Jul. 16, 2003.
(60) Provisional application No. 60/397,653, filed on Jul. 22, 2002.

(51) Int. Cl. A61B 8/14

(52) U.S. Cl. 600/433

(57) ABSTRACT

An intra-cardiac imaging system that includes an ultrasound catheter that can image the left heart from within the right heart. The catheter has a proximal end, a distal end, and a lumen extending therebetween. The distal end includes an acoustic window longitudinally oriented and having a length of at least ten millimeters. A linear ultrasound transducer having an active surface is longitudinally mounted inside the lumen of the catheter at the distal end of the catheter adjacent the acoustic window. The active surface of the ultrasound transducer is directed toward the acoustic window, is approximately the same length as the acoustic window, and is capable of transmitting an ultrasound signal at a frequency of about 1.5 MHz to about 9 MHz.

### RELATED APPLICATION

This is a continuation-in-part of U.S. patent application Ser. No. 10/620,517 (Attorney Docket No. 79147; US Publication No. 2004/0127798 A1), filed on Jul. 16, 2003, which claims benefit of U.S. Provisional Application Ser. No. 60/397,653, filed on Jul. 22, 2002, both of which are herby incorporated by reference.

### BACKGROUND OF THE INVENTION

1. Field of the Invention

The present invention relates generally to methods of ultrasound imaging, and more particularly ultrasound imaging of the left heart from within the right heart, and also to ultrasound imaging catheters that can image the left heart from within the right heart.

2. Description of the Related Art

Volumetric output of blood from the heart and/or circulatory system are of interest in various diagnostic and therapeutic procedures. Such measurements are of significant interest during electrophysiological evaluation/therapy to first evaluate the extent of cardiac dysfunction due to arrhythmia and subsequently to judge the effect/effectiveness of any ablations/therapeutic procedures that are carried out on the cardiac muscle/conduction system. Iwa et al., Eur. J. Cardithorac. Surg., 5, 191-197 (1991).

Ultrasound is the imaging modality of choice, especially in cardiology, since this modality offers real-time imaging capabilities of the moving heart. Further, advances through Doppler techniques allow the physician to visualize as well as measure blood flow. Pulse wave and continuous wave Doppler have proven to be quite accurate, and an effective way of evaluating flow through various parts of the circulatory system, especially the heart. Tortoli et al., Ultrasound Med. Bio., 28, 249-257 (2002); Mohan et al., Pediatr. Cardiol. 23, 58-61 (2002); Ogawa et al., J. Vasc. Surg., 35, 527-531 (2002); Pislaru et al., J. Am. Coll. Cardiol., 38, 1748-1756 (2001).

Other technologies, including washout curves of contrast agents have been proposed to measure flow volume, especially to compensate for loss of signal quality due to imaging depth. Krishna et al., Ultrasound Med. Bio., 23, 453-459 (1997); Schrope et al., Ultrasound Med. Bio., 19, 567-579 (1993).

However, until recent advances in miniaturized ultrasonic transducers, physicians were limited to only certain angles of view, thus limiting the range and effectiveness of possible measurements. Further, given the depth of imaging required by such classical approaches, associated interrogation frequency limitations due to attenuation restricted the accuracy of measurements. Krishna et al., Phys. Med. Biol., 44, 681-694 (1999). With the recent introduction of catheter based ultrasound transducers for imaging the heart from the vena-cava or from within the heart, such limitations on frequency of interrogation and angle of view are no longer applicable.

Catheters for insertion and deployment within blood vessels and cardiac chambers are well-known in the art. Physicians have been using intra-cardiac ultrasound catheters, for example, to obtain visual guidance during procedures, such as intracardiac echocardiography, pulmonary vein ablation, transcatheter septal defect closures, identifying anatomic abnormalities before therapeutic procedures, visualizing the relative orientation of diagnostic and therapeutic catheters, pacemaker or defibrillator lead insertion or extraction, transseptal catheterization, valvuloplasty, and balloon septostomy.

Insertion of catheters into the heart during such procedures has generally been limited to the venous, or right side of the heart. The reason for this is that surface imperfections, for example, can cause blood clots or other emboli formation in some patients. If a blood clot or embolus were released arterially from the hearts' left side, as for example the left ventricle, it could pass directly to the brain potentially resulting in paralysis or a fatal stroke. However, a blood clot or embolus released from the right heart, as from the right ventricle, would pass into the lungs where the filtering action of the lungs would prevent a fatal or debilitating embolism in the brain.

To avoid such devastating consequences as stroke, intra-cardiac ultrasound imaging catheters, such as electrophysiology catheters with ultrasound transducers, are generally introduced into the right heart, through either the superior or inferior vena cava and into the right atrium. Current ultrasound catheters typically have an imaging depth of a few centimeters. The consequent limitation is that only the right heart can be adequately imaged from the right atrium.

The human heart, in many diseased conditions, enlarges to dimensions wherein points closer to the apex of the heart, especially on the left side, are over 15 cm away from the vena cava—left atrium junction. Therefore, imaging at over 15 cm imaging depth is necessary for full-fledged use of intra-cardiac imaging.

One specific need for extended ultrasound imaging depth is for the permanent placement of cardiac pacing electrodes. Cardiac pacing has been around for many years, and essentially involves the placement of a permanent electrode in the right ventricle to coordinate the contraction of the ventricle with the atria. A new therapy has recently been introduced to the market, which involves pacing of the left ventricle in conjunction with the right ventricle in an effort to “resynchronize” the heart, that is, to coordinate the left ventricle's contraction in time with the contraction of the right ventricle. One problem in the current therapy is the optimization of the placement of the left ventricular electrode so as to provide maximum therapy. Thus, there is a need for intracardiac ultrasound imaging catheters which can image the left heart from the right heart to aid in electrode placement in the left heart.

Therefore, a need exists for ultrasound catheters with improved imaging capabilities, particularly increased depth of view to image distant anatomical structures such as the left heart from within the right heart.

### SUMMARY OF THE INVENTION

Provided herein is an intra-cardiac imaging system that includes an ultrasound catheter that can image the left heart from within the right heart. The catheter has a proximal end, a distal end, and a lumen extending therebetween. The distal end includes an acoustic window longitudinally oriented and having a length of at least ten millimeters. A linear ultrasound transducer having an active surface is longitudinally mounted at the tip of the catheter at the distal end of the catheter. The ultrasound transducer is capable of transmitting an ultrasound signal at a frequency of about 1.5 MHz to about 9 MHz. The catheter can further include one or more pacing electrodes and/or one or more defibrillation electrodes.

### BRIEF DESCRIPTION OF THE DRAWINGS

FIG. 1 provides a general system diagram showing an ultrasound system.

FIGS. 2A, 2B, and 2C provide various embodiments of the present system with an attached workstation.

FIG. 3 provides diagrams of a typical B-mode image and an associated Doppler spectrum. A cross-sectional view of the ventricle and the aortic value are shown as viewed from the right atrium. The spectral Doppler waveform shows the velocity profile of the flow at the aortic valve.

FIG. 4 provides a genera diagram illustrating the basic technique used to measure volume of flow from a spectral Doppler spectrum, and the approximate correlation of the ECG with the Doppler spectrum readout. The flow being samples taken at the aortic valve (as shown in FIG. 3). Multiple peak velocity points can be utilized as shown in the first and second Doppler waveforms with increasing numbers of points providing increased accuracy.

FIG. 5 provides a diagram illustrating the measurement technique for calculating cross-sectional area of the output from the ventricle. In this view, the ultrasound catheter is positioned in the vena-cavae or in the right atrium. Other anatomical locations for placement of the ultrasound catheter can, of course, be used.

FIG. 6 illustrates the basis of Doppler measurement used in an embodiment of the present invention by delineating streamlined flow through a vessel, its profile through time and the basis of the time-integral area product showing volume of flow.

FIG. 7 illustrates the basis of M-mode measurement used in an embodiment of the present invention. Two walls of the ventricle are viewed using M-mode. One cross section is shown relative to the associated electrocardiogram.

FIG. 8 provides a perspective view of an ultrasound system for use in an embodiment of the present invention including the ultrasound console, connecting isolation box, and the ultrasound catheter. The isolation box provides electrical isolation between the patient and the ultrasound system as required by current FDA guidelines.

FIG. 9 generally illustrates a normal heart (i.e., non-congestive failure (CHF) heart). Panel A illustrates the right atrium (RA), left atrium (LA), right ventricle (RV), and left ventricle (LV) as well as the location of an electrode (“lead”) placed on the right ventricle to provide electrical pulses to the heart; the directions of the normal pacing pathways are also shown. Panel B illustrates the direction of normal contraction of the heart muscle in the ventricles.

FIG. 10 generally illustrates a CHF heart with enlargement of the left ventricle. Panel A illustrates the enlargement of the left ventricle normally observed with CHF; the dotted line in the left ventricle is included to illustrate the normal heart (i.e., non-CHF) as shown in FIG. 9. Panel B generally illustrates the area slow conduction and the normal area for placement of an electrode for re-synchronization. Panel C generally illustrates the direction of potential contraction normally associated with CHF without re-synchronization.

FIG. 11 generally illustrates placement of the ultrasound catheter of this invention in the right ventricle to image the left ventricle according to an embodiment of the present invention.

FIG. 12 provides computer flowcharts illustrating the procedures for estimating cardiac output using Doppler based techniques (Panel A) and M-mode based techniques (Panel B).

### DETAILED DESCRIPTION

Heart failure is a disease where the heart's main function, a pump for blood, is not optimal. The left ventricle does not allow quick electrical conduction, becomes enlarged, does not contract well, and becomes less efficient at pumping blood. A measurement for the efficiency of the heart as a pump is called “ejection fraction” or “EF”. EF is measured as the percentage of blood contained in the ventricles that is pumped out with each beat of the heart. A healthy, young heart will have an EF greater than 90% (i.e., 90 percent of the ventricular blood is pumped with each heart beat); an older, sick heart in heart failure can have an EF less than 30%. Heart failure leads to an extremely diminished lifestyle, and, left untreated, can be a major cause of mortality.

A new therapy to treat heart failure is bi-ventricular pacing, or “resynchronization” therapy, where both ventricles of the heart are paced with an implantable pulse generator, commonly known as an artificial pacemaker. Normal pacing for a slow heart is performed via an implanted electrode in the right ventricle. The conduction myofibers (Purkinje fibers) conduct the electrical pulse and the ventricles contract synchronously in an inward direction, resulting in blood being pumped efficiently from the heart. In heart failure, the left ventricle becomes enlarged and conduction through the tissue of the left ventricular wall often becomes slow, so that the upper part of the left ventricle contracts as much as 200 to 250 milliseconds after the apex area of the ventricles contract. This leads to poor and discoordinated contraction, and in many cases, an outward movement of the heart muscle, so that blood sloshes around inside the ventricle rather than being squeezed out of the ventricle. Thus, an ideal location to place a pacing electrode in the left ventricle is in the area of slowest conduction, which can be a rather large area of the left ventricle, and may not always be the area that has the largest contraction. The problem facing physicians today is to locate the optimal spot for the permanent fixation of the pacing electrode. An embodiment of the present invention provides a method and device to optimize the location of the electrode.

A normal pacemaker electrode is ideally implanted in a location which achieves the lowest “threshold,” which is the lowest voltage level to excite the surrounding tissue to synchronously conduct the pacing signal from the electrode. Thus, the electrode is implanted based upon merely finding the spot with the lowest voltage that “captures” the tissue. With heart failure, in the left ventricle, it is not so simple. Capture may not be the best parameter to use. Furthermore, advancing the electrode to the proper spot may not be easy. What is most desired is to optimize EF, while the threshold for “capture” is really secondary. Thus the ability to not only visualize the motion of the left ventricular wall, but also measure EF, or some form of output of the heart, such as stroke volume or flow rate, is highly desirable during the implantation procedure. This invention puts forth the use of ultrasound technology for this purpose.

The present invention is directed to a method and system for measuring volumetric flow, specifically cardiac output, either with minimal intervention/input from the physician, or automatically, with the user of the system pre-specifying certain operating parameters/measurement criterion. One embodiment of the present invention is in the form of hardware and/or software that exists as part of the ultrasound scanner. In such an embodiment, the system utilizes the Doppler processing capabilities of the host ultrasound scanner to obtain a time-varying signal representative of the velocity of flow through an area of interest. Such area could include the inlet of the aorta from the left ventricle, or the valve in between. The system also utilizes a view/measure of the cross-sectional area through which the flow of interest is to pass.

Measurements of blood flow using information extracted from the Doppler frequency shift of ultrasound echoes received by an ultrasound probe (“Doppler signals”) may be used to calculate volume of blood flow through an imaged area. Such calculations may employ the Doppler signals, the boundaries of which can be either demarcated by the user, or automatically estimated by the system, and the measured cross-sectional area through which such flow passes, which can again be either demarcated/input by the user, or can be automatically measured by the system. This information is utilized by the processor, or any other hardware, software, or combination thereof, to calculate volume of flow through the area of interest.

Other embodiments also include the measuring system, either in the form of software and hardware or a combination thereof on a separate workstation/computer that is capable of obtaining relevant data from the examining ultrasound scanner either directly or indirectly, and methods of being triggered/correlating the ultrasonic/Doppler signals (video/audio) with the electrocardiogram (ECG) of the subject being examined.

Another embodiment of the present invention utilizes the Doppler audio output of the Doppler processing system/sub-system in the ultrasound machine in addition to the facilities to obtain the measure of the area of interest through which the flow is to pass, and the ECG of the subject being examined. Again, this process/system can be embodied within the hardware and/or software of the ultrasound scanner, or implemented as a workstation and/or computer separate from the ultrasound scanner with facilities to communicate either directly or indirectly with the ultrasound scanner. Such processing then uses the frequency, phase, and amplitude of the audio signals along with the measure of the area of interest through which the flow exists to calculate the volume of flow. A further embodiment can also include methods of obtaining ECG data from the subject being scanned to enhance the demarcation and/or separation of signals from beat to beat of the heart, or to assess either automatically, or aided by a user, the condition of the cardiac system and hence the factors effecting the acquired Doppler data.

The M-mode based embodiment would include hardware and/or software, either on the ultrasound system, or on a separate system that directly or indirectly communicates/receives data from the ultrasound system and a device that can digitize and/or transmit ECG data, if separate from the ultrasound unit. This device would then utilize these signals, in coordination with the ECG signals to calculate the spacing between the walls of the left ventricle to obtain the maximum and minimum volumes of the ventricle in the course of a cardiac cycle.

Ultrasound, as an imaging tool, has been around for some time. However, imaging through the chest is very difficult because the ribs block the view to the heart and that the depth of penetration gives poor resolution. Ideally, the ultrasound transducer should be positioned closer to the heart. An esophageal ultrasound probe has been used on more than 50 patients in an attempt to view the heart. See, e.g., Jan et. al., Cardiovasc. Intervent. Radiol., 24, 84-89 (2001). Unfortunately, the results are less than desired since the probe must view through the esophagus and both walls of the heart, lending to less resolution in the image than desired. Intravascular ultrasound systems, although ideal in its size with thin catheters, generally utilize high frequencies which result in poor depth of penetration. X-ray imaging or X-ray fluoroscopy may give good images of the electrode, but not of the actual tissue of the heart (most particularly the walls of the ventricle).

The present invention overcomes one or more of these problems. Preferably, an embodiment of the present invention uses an ultrasound imaging catheter designed for intracardiac use. Such an intracardiac catheter is generally sized to be about 10 French or less, has multiple elements on the transducer (e.g., 48 or 64 elements), employs lower frequencies (e.g., between about 1 and about 10 MHz, and more preferably between about 1.5 and 9 MHz), uses a phased array transducer for optimal resolution, and has an acoustic window of about ten millimeters or more in length. Not only will this allow the imaging of wall motion for the specific purpose of a left ventricular electrode fixation, but will also, especially if used in conjunction with Doppler techniques, provide information to calculate measurement of cardiac output.

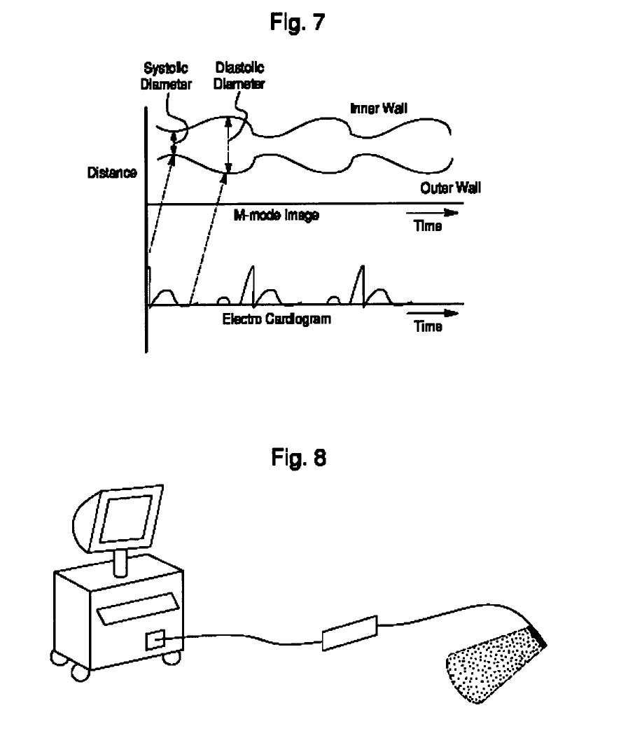

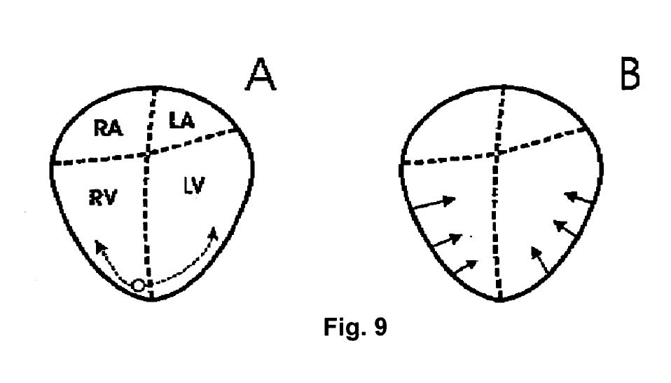

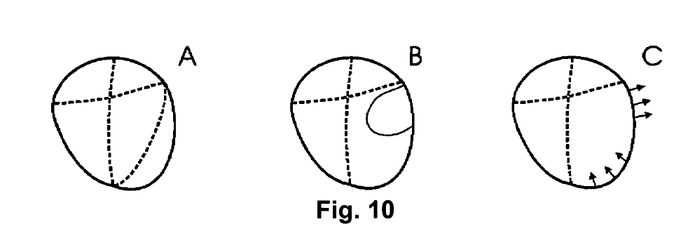

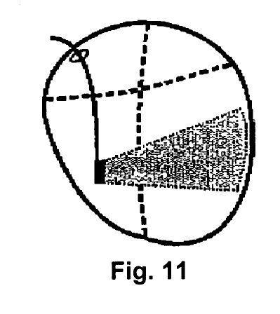

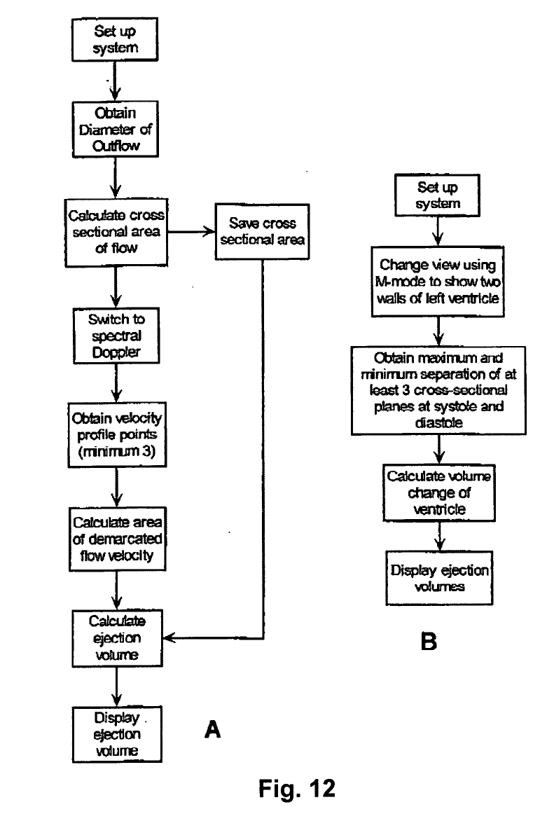

Such a catheter could be placed in either the right atrium of the heart or the right ventricle and easily allow viewing of the left ventricle (FIG. 7). Another approach for viewing could be from the outside of the heart, via an incision through the chest of a patient. This catheter would connect either directly to a display system or through a connecting cable, as shown in FIG. 6. The ultrasound display can provide a display of the measurement of cardiac output in assisting the physician with the procedure.

In addition to ultrasound imaging, a number of other items may make this implant an easier procedure, especially since many of the heart failure physicians may not have previously implanted pacemakers, may not have access to x-ray fluoroscopy, may have limited budgets for capital equipment, and may desire all discreet components used in an implantation to be accessible through one keyboard, allowing for better patient data management. Some of these improvements include:

1. Combining the ultrasound with a robust cardiac electrophysiology recording device such that both surface electrocardiograms and internal electrocardiograms can be recorded and displayed. Both electrograms, while not necessary, could substantially assist in the procedure.

2. The left ventricle electrode can be implanted in a spot chosen by imaging as well as voltage mapping. An overlay of these two parameters could more easily allow the physician to visualize the mechanical and electrical characteristics at the same time.

3. Often times the heart failure patient has a number of co-morbidities showing symptoms at the same time, such as atrial fibrillation, ventricular tachycardias, and renal failures, among others. Atrial fibrillation and ventricular tachycardia can be brought under control via electrical shock cardioversion, either internally with catheters, or externally, although with much higher energy, with patches or paddles. A cardioversion device which could utilize the same electrodes that are otherwise introduced into the heart for pacemaker implantation, would be advantageous if also integrated with the overall electrophysiology system. In this manner, inadvertent shocks could be avoided as the trigger mechanism would come from the ventricular signal from the internal electrode. Thus, in one embodiment, the ultrasound imaging system of the present invention also comprises an integral defibrillation system whereby, if needed, internal cardiac defibrillation can be implemented quickly and easily. The integrated defibrillation electrode or system may be incorporated into the ultrasound imaging catheter, attached to the ultrasound imaging catheter, or as a separate electrode system or catheter which is inserted along with the ultrasound imaging catheter.

The present invention provides an ultrasound imaging system suitable for measuring cardiac output of a patient's heart, said system comprising:
(1) an ultrasound imaging catheter comprising at least one transducer utilizing piezoelectric properties to generate acoustic signals from electrical signals in order to obtain ultrasound signals, wherein the at least one transducer is suitable for insertion into the patient's heart and to obtain ultrasound signals associated with an area of the patent's heart in which cardiac output is to be measured;
(2) digital and/or analog electronics capable of generating and processing ultrasound signals from the at least one transducer to generate B-mode, M-mode, or Doppler representations of the cardiac output of the patient's heart; and
(3) an associated computer that can generate and process the ultrasound signals in order to measure the cardiac output in the patient's heart.

This invention also provides a method of placing an electrode at a desired position at or near the left ventricle of a patient's heart in order to electrically activate the left ventricle of the patient's heart using the electrode, said method comprising:
(1) advancing the electrode to the proximity of the upper left ventricle;
(2) placing an ultrasound imaging catheter in a position to image the left ventricle of the patient's heart, wherein the ultrasound imaging catheter comprises at least one transducer utilizing piezoelectric properties to generate acoustic signals from electrical signals in order to obtain ultrasound signals and wherein the at least one transducer is suitable for insertion into the patient's heart and to obtain ultrasound signals associated with an area of the patent's heart;
(3) utilizing the ultrasound imaging catheter to image the electrode at or near the left ventricle of a patient's heart and to guide the electrode to the desired position; and
(4) attaching the electrode to the desired position. One preferred desired position for attachment of the electrode is the upper portion of the left ventricle (i.e., nearer the base of the heart as compared to the apex). In one preferred embodiment, at least one transducer has a defecting or rotation element whereby the transducer, once positioned to image the left ventricle of the patient's heart, can be easily rotated or moved in order to image other portions of the patient's heart.

The present invention also provides an ultrasound imaging system to assist in cardiac electrophysiology procedures related to a patient's heart, said system comprising:
(1) an ultrasound imaging catheter comprising a multi-element array transducer utilizing piezoelectric properties to generate acoustic signals from electrical signals in order to obtain ultrasound signals, wherein the multi-element array transducer is suitable for insertion into the patient's heart and to obtain ultrasound signals associated with the patent's heart;
(2) digital and/or analog electronics capable of generating and processing ultrasound signals from the multi-element array transducer to generate and display a representation of (a) the electrocardiogram of the patient's heart, (b) a real time image of the patient's heart, or (c) the cardiac output of the patient's heart. In a preferred embodiment, the representation ultrasound signals can be displayed relative to, and compared to, a voltage conduction map of the patient's heart (i.e., a representation of the progression of electrical activation/deactivation or “action potentials” of the muscles of the heart).

The basis of the measurement/estimation process of various embodiments of the present invention is shown in FIGS. 6 and 7. Using the Doppler process (FIG. 6), the amplitude of the velocity profile is halved to provide the average velocity across the flow area (FIG. 6A). The velocity is integrated (FIG. 6B) with respect to time from the start of the pulse (t0) to the end of the pulse (t1). Such integration can also include the negative peaks shown in FIG. 4A to compensate for reverse flows. The result of this integration with respect to time is then multiplied by the cross-sectional area of the flow to provide the ejection volume (FIG. 6C). The integration length can also be set by integrating during the complete cardiac cycle (i.e., through one complete cycle of the ECG). The spectrum in FIG. 6 can also be obtained by either frequency and/or amplitude plotting of an audio signal.

$${V}_{ejt} = A \int \frac{{V}_{peak}}{2} ⅆ t \qquad Eq . 1$$

where Vₑⱼₜ=Ejection volume/stroke volume;
A=cross sectional area of flow; and
Vₚₑₐₖ=points on the velocity curve.

Using the M-mode process (FIG. 7), the system outputs the relative position of the two walls of the ventricle as a function of time. The ventricle can be equated to an ellipsoid shape, whose secondary radius is represented by the distance between the two walls measured by the M-mode. The primary equation to the volume would then be
V=(π(R₁+C₁)R₂)(2πR₂)±C₂ Eq. 2
where V=volume
R₁=Primary radius=length of the ventricle;
R₂=secondary radius=distance between the walls of the ventricle;
C₁=a correction factor to compensate for the difference in morphology of the ventricle w.r.t. an ellipse; and
C₂=correction in the primary radius to compensate for longitudinal contractility of the ventricle during a cardiac cycle.

Volume can then be calculated at systole and diastole (determined either with correlation to the ECG, as shown in FIG. 7 or by determining the minimum and maximum of the M-mode curve). The stroke volume is then given by
Vₛᵥ=V_{diastole}−V_{systole} Eq. 3.

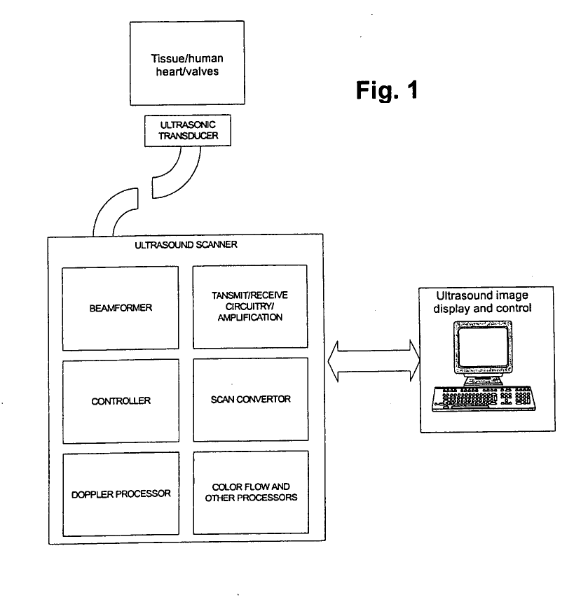

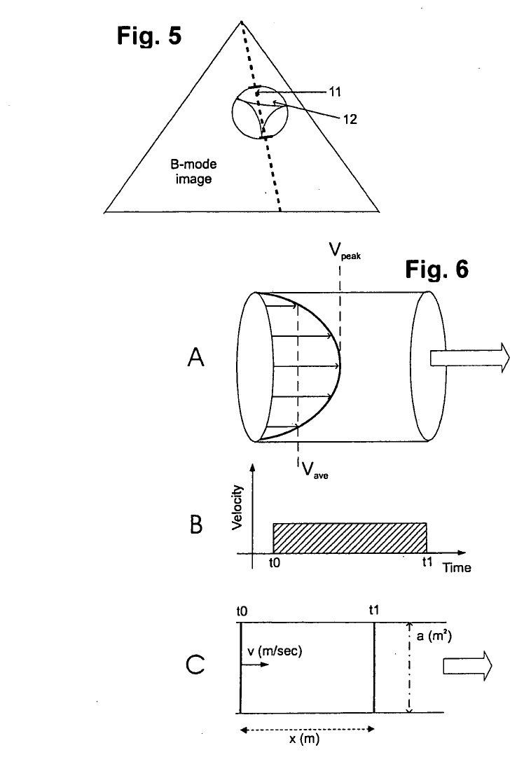

One embodiment of the present invention is in the form of hardware and/or software that exists as part of the ultrasound scanner (FIG. 1). In such an embodiment, the system utilizes the Doppler processing capabilities of the host ultrasound scanner to obtain a time-varying signal representative of the velocity of flow through an area of interest. Such area could include the inlet of the aorta from the left ventricle, or the valve in between. The system also utilizes a view/measure of the cross-sectional area through which the flow of interest is to pass (FIG. 5).

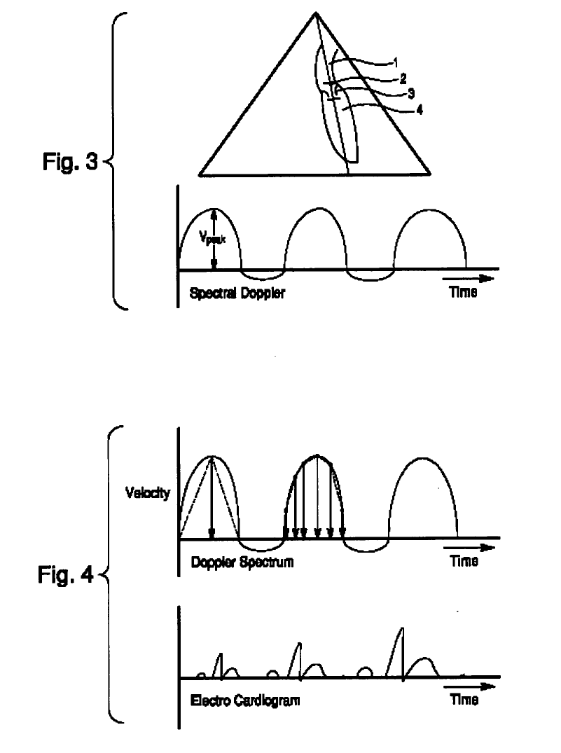

The Doppler system outputs the spectral information, which is indicative of the velocity of flow through the volume of interest (as shown in FIG. 3) either by means of showing a spectrum (which in some embodiments can be obtained in a analog or digital format from the machine). Such a spectrum can be obtained either by obtaining a longitudinal sectional view of the flow axis at any angle (as represented in FIG. 3), or by obtaining a cross sectional view of the flow conduit (FIG. 5). Such calculations of flow/area can be compensated for the angle of measurement using a cosine of the angle w.r.t. actual plane correction. For conditions where the flow is perpendicular to the sample volume of the Doppler system, other estimation techniques such as “Transverse Doppler,” which utilizes the Doppler bandwidth to assess flow at flow to beam angles close to 90 degrees, can be utilized. Tortoli et al., Ultrasound Med. Biol., 21, 527-532 (1995). This Doppler signal can also be as an audio signal (again, either in analog or digital format) as a frequency and/or amplitude modulated signal that is indicative of the spectrum and hence the flow velocity through the area of interest. This could further include ECG signals (again, in analog or digital format).

Further processing can be carried out, for example, using the following techniques:

1. A largely manual process wherein the user measures/demarcates, either with or without the aid of an ECG, the peak velocities at least one point on the spectrum and demarcates/measures the cross-section of the outlet of the ventricle; and the system/calculating tool (either on the ultrasound machine or on a separate computer) the integrates the curve over time to obtain stroke volume via Equation 1.

2. A semi-automated process wherein the system (either on the ultrasound machine or separate) automatically integrates the curve with or without the help of an ECG while the user inputs the area of interest of the orifice through which the flow passes.

3. A fully automated process wherein the system prompts the user to obtain particular views of the anatomy of interest and demarcate specific points and the system then processes the data as above with, however, the system internally tracking the data of interest.

4. The system automatically integrates the curve from beat to beat, and outputs the stroke volume in any sort of display, having obtained the cross sectional area using the techniques mentioned in point 2 or 3 above. Of course, various combinations and/or modifications of these techniques can be used if desired and depending on the particular application and/or patient.

Another embodiment of the present invention is in the form of hardware and/or software that exists separate from the ultrasound scanner console or workstation with means to communicate either video and/or audio and/or other signals between the ultrasound scanner and/or the display computer/system. Communication between such workstation and the ultrasound scanner could include video, audio, and/or any ECG signals in digital and/or analog format. The above described processing can then be performed either partially or entirely on the workstation.

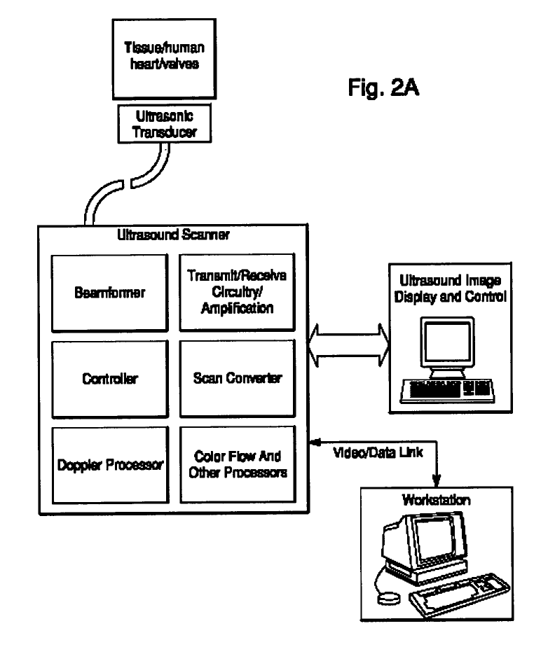

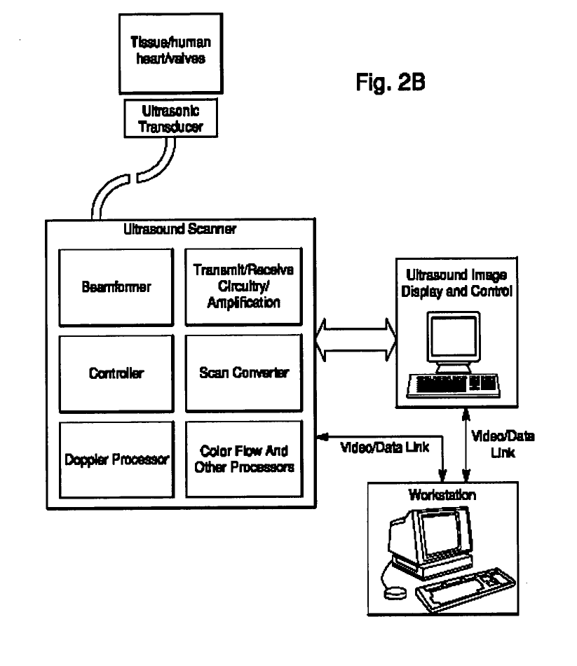

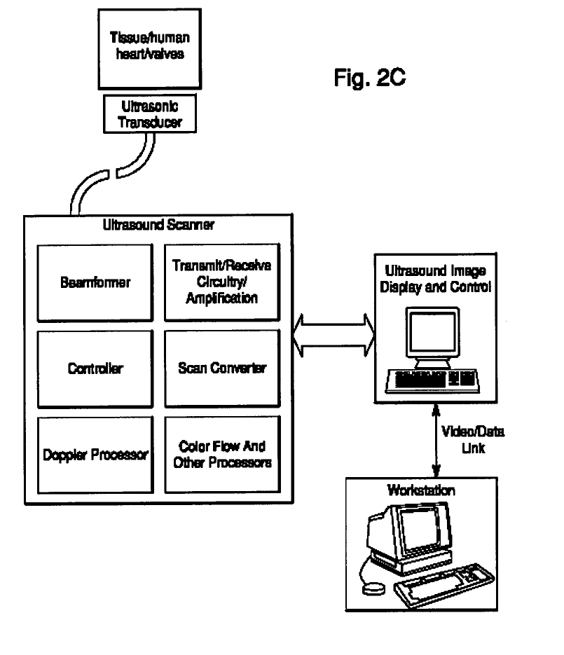

In another embodiment of the present invention, the M-mode output is utilized to measure stroke volume. Again, this system can comprise hardware and/or software that resides wholly on the ultrasound scanner or can also include hardware and/or software on a separate workstation with means to communicate either digital and/or analog data with the ultrasound scanner (FIGS. 1 and 2). The volume can then be estimated, as given earlier by Equations 2 and 3 (FIG. 7).

Processing can be carried out, for example, using the following techniques:

1. A largely manual process wherein the user measures/demarcates, either with or without the aid of an ECG, the systolic and diastolic distances between the two ventricular walls, and the system/calculating tool (either on the ultrasound machine or on a separate computer) calculates the stroke volume. This process can include, if desired, provisions for the user or system to record/obtain the correction factors described in Equation 2.

2. A semi-automated process wherein the system (either on the ultrasound machine or separate) automatically measures the distances and estimates the stroke volume with or without the help of an ECG. In this case, the system can automatically measure/estimate the correction factors described in Equation 2, or the user can specify or aid the system in estimating/measuring these factors.

3. A fully automated process wherein the system prompts the user to obtain particular views of the anatomy of interest and demarcate specific points and the system then processes the data as above with, however, the system internally tracking the data of interest.

4. The system automatically measures the stroke volume, with data obtained from any of the above described methods, and outputs the stroke volume in any sort of display, having obtained the cross sectional area using the techniques mentioned in points 2 or 3 above.

Yet another embodiment can include hardware and/or software separate from the ultrasound scanner, in the form of a workstation wherein there exists a mode of communication, either analog or digital, between the workstation and the ultrasound scanner or catheter. Cabling from the ultrasound machine to the catheter (especially with a multi element array catheter) and from the catheter proximal connector to the catheter transducer housed at the distal tip can be expensive. To reduce cost, the ultrasound machine could be moved adjacent to the patient, thereby allowing a relatively short cable to be used to attach the catheter. In some cases, however, this may be impractical since most catheter rooms are sterile or semi-sterile environments and, thus, the ultrasound machine may be some distance from the patient's bedside. Thus, a connecting cable which is reusable (and probably non-sterile) is desirable, as opposed to the catheter itself which is sterile and usually not re-usable. While many ultrasound machines have a standard 200 pin ZIF connector, most ultrasound machines do not have patient isolation means built in to the degree necessary for percutaneous catheter use. Therefore, in another embodiment, the system of this invention employs a connector cable with an isolation means or isolation box that is external to the ultrasound machine itself. Preferably the isolation box, which houses a plurality of isolation transformers, is relatively small so that it could be placed easily on or near the patient's bed. Such a cable could easily accommodate all operational communication between the catheter and the ultrasound machine and/or the appropriate computer workstation.

In still another embodiment, the ultrasonic catheter further comprises a temperature sensing and/or control system. Especially when used at higher power (e.g., when using color Doppler imaging) and/or for lengthy periods of time, it is possible that the transducer, and hence, the catheter tip, may generate heat that may damage tissue. While computer software can be used to regulate the amount of power put into the catheter to keep the temperature within acceptable ranges, it is also desirable to provide a temperature sensing means as well as a safety warning and/or cut-off mechanism for an additional margin of safety. Actual temperature monitoring of the catheter tip is most desirable, with feedback to the computer, with an automatic warning or shut down based upon some predetermined upper temperature limit. The system could be programmed to provide a warning as the temperature increases (e.g., when it reaches 40° C. or higher) and then shut off power at some upper limit (e.g., 43° C. as set out in U.S. FDA safety guidelines). To monitor the temperature at or near the tip of the catheter (i.e., in the region of the ultrasound transducer), a thermistor may be used. The temperature at the tip of the catheter could be continuously monitored via appropriate software. Although the software could also provide the means to control the power to the catheter in the event that excessive temperatures are generated, it would also be desirable to have a back up shut off or trip mechanism (e.g., a mechanical shut off or tripping means).

In yet another embodiment, as shown in FIG. 13, a catheter 700 is shown having a proximal end 710, a distal end 720 and lumen 730 extending therebetween. The lumen 730 carries the cables (not shown) that connect the transducer 750 located at the distal end 720 of the catheter 700 to the ultrasound controller or console 800 (shown in FIG. 14). The catheter 700 also includes one or more pacing electrodes 760 and one or more defibrillation electrodes 770. Alternatively, the pacing electrodes 760 could also function as defibrillation electrodes, thus obviating the need for additional defibrillation electrodes. The catheter 700 also includes a linear ultrasound transducer 750 having an active surface 755 that is directed toward an acoustic window 780 formed in the catheter body at the distal end 720 of the catheter 700. The linear transducer 750 is longitudinally mounted inside the lumen 730 of the catheter 700 at the distal end 720 of the catheter 700 adjacent the acoustic window 758. The transducer 750 is approximately the same length as the acoustic window 758, and is capable of transmitting an ultrasound signal at a frequency between about 1 MHz and about 10 MHz, and more preferably between about 1.5 MHz and about 9 MHz. The lower frequencies can penetrate deeper into the left heart. The acoustic window is at least approximately 10 millimeters in length to improve penetration into deep or distant anatomical structures. Specifically, as with any antenna that emits radiation—the wider the antenna the more sensitive it is. Such an acoustic window may have any number of shapes, such as rectangular, gaussian, and/or Hamming. Further, any single or combination of materials that allow impedance matching between the material of the transducer and surrounding tissue can be used to fabricate the acoustic window, including some types of Silicone.

The ultrasound catheter 700 is intended for placement in the right heart for imaging the left heart. The ultrasound capabilities must, therefore, enable imaging anatomical structures that are 15 cm or more from the transducer 750. Various electronics are incorporated into the catheter imaging system to allow for imaging distant anatomical structure. As shown in FIG. 14, these include one or more in line buffer amplifiers 810 and one or more transmit-bypass circuits 830. The ultrasound controller 800 sends an electrical signal 805 that is translated into a center frequency of between about 1 MHz and about 10 MHz by the transducer 750, and more preferably between about 1.5 MHz and about 9 MHz. An in line buffer amplifier 810 and transmit-bypass circuit 830 located between the transducer 750 and controller 800 compensates for line attenuation and improves signal to noise ratio. The transmit-bypass circuit 830 reduces the risk of damage to the in line buffer amplifier 810 that could be caused by the power amplifier 820 that amplifies the electrical signal 805 en route to the transducer 750. The echoing ultrasound waves that are received by the transducer are weaker than the waves that were transmitted by the transducer 750. Thus, the electrical signal 807 transmitted by the transducer 750 is a weaker signal. The in line buffer amplifier 810 amplifies the signal 807, thus compensating not only for the reduced signal 807 but also for line attenuation. The in line amplification could be a simple buffer amplifier using a high CMRR OpAmp, with one amplifier for each ultrasound line. Other configurations are also contemplated.

Of course, various combinations and/or modifications of these techniques and systems can be used if desired and depending on the particular application and/or patient.

It is to be understood, however, that even though numerous characteristics and advantages of the present invention have been set forth in the foregoing description, along with details of the structure and function of the invention, the disclosure is only for illustrative purposes. Changes may be made in detail, especially in matters of shape, size, arrangement, and storage/communication formats within the principles of the invention to the full extent indicated by the broad general meaning of the terms in which the appended claims are expressed.

1. An intra-cardiac imaging system comprising:
a catheter having a proximal end, a distal end, and a lumen extending therebetween, the distal end comprising an acoustic window longitudinally oriented and having a length of at least ten millimeters; and
a linear ultrasound transducer having an active surface, the linear transducer longitudinally mounted inside the lumen of the catheter at the distal end of the catheter adjacent the acoustic window, wherein the transducer is capable of transmitting an ultrasound signal at a frequency of about 1.5 MHz to about 9 MHz.

2. The intra-cardiac imaging system of claim 1, wherein the active surface of the linear transducer is directed toward the acoustic window and is approximately the same length as the acoustic window.

3. The intra-cardiac imaging system of claim 1, further comprising:
an inline buffer amplifier that receives a signal from the ultrasound transducer and amplifies it; and
an ultrasound imaging console that receives the signal from the inline buffer amplifier.

4. The cardiac imaging system of claim 3, further comprising a transmit-bypass circuit between the catheter and console.

5. A intra-cardiac catheter comprising:
a shaft having a proximal end, a distal end, and a lumen extending therebetween, the distal end comprising an acoustic window longitudinally oriented and having a length of at least ten millimeters; and
linear ultrasound transducer having an active surface, wherein the active surface of the linear transducer is directed toward the acoustic window and is approximately the same length as the acoustic window, and wherein the transducer is capable of transmitting an ultrasound signal at a frequency of about 1.5 MHz to about 9 MHz.

6. A method of imaging a left side of a heart of an individual from a right side of the heart comprising:
providing a catheter comprising:
a proximal end, a distal end, and a lumen extending therebetween, the distal end comprising an acoustic window longitudinally oriented and having a length of at least ten millimeters; and
a linear ultrasound transducer having an active surface, the linear transducer longitudinally mounted inside the lumen of the catheter at the distal end of the catheter adjacent the acoustic window, wherein the transducer is capable of transmitting an ultrasound signal at a frequency of about 1.5 MHz to about 9 MHz;
making an incision in the individual;
inserting the catheter through the incision;
advancing the catheter into the right atrium of the heart; and
activating the transducer to transmit an ultrasonic pulse toward the left side of the heart, the ultrasonic pulse having a frequency of about 1.5 MHz to about 9 MHz.

7. The method of claim 6, further comprising:
receiving one or more reflected ultrasound waves from the left side of the heart;
using the transducer to transform the one or more reflected ultrasound waves into an electrical signal;
using an amplifier to amplify the electrical signal; and
displaying an image representative of the amplified signal on a monitor.

8. A method of intra-cardiac ultrasound imaging, comprising:
transmitting an ultrasonic pulse toward a left side of a patient's heart;
receiving, in a right side of the patient's heart, one or more reflected ultrasound waves from the left side of the heart; and
displaying an image representative of the received ultrasound waves.

9. The method of claim 8, wherein the one or more reflected ultrasound waves reflect from an object at least 15 cm away from a source of the ultrasound pulse.

10. The method of claim 8, further comprising generating a signal representative of the received ultrasound waves.

11. The method of claim 10, further comprising amplifying the signal with an in-line amplifier.

12. The method of claim 8, wherein the ultrasonic pulse has a frequency of about 1.5 MHz to about 9 MHz

13. An intra-cardiac ultrasound imaging system, comprising:
means for transmitting an ultrasonic pulse within a right side of a patient's heart and toward a left side of the patient's heart;
means for receiving one or more reflected ultrasound waves from the left side of the heart within the right side of the heart; and
means for displaying an image representative of the received ultrasound waves.

14. The intra-cardiac ultrasound imaging system of claim 13, wherein the ultrasonic pulse has a frequency of about 1.5 MHz to about 9 MHz

15. The intra-cardiac ultrasound imaging system of claim 13, wherein the one or more reflected ultrasound waves reflect from an object at least 15 cm away from the means for receiving.

16. The intra-cardiac ultrasound imaging system of claim 13, further comprising means for generating a signal representative of the received ultrasound waves.

17. The intra-cardiac ultrasound imaging system of claim 16, further comprising means for amplifying the signal.
